(19) United States
(12) Patent Application Publication
(10) Pub. No.: US 2005/0004519 A1
(43) Pub. Date: Jan. 6, 2005

(54) Ear syringe tip

(76) Inventor: Werner Desmond Van Jaarsveldt, Winnipeg (CA)

Correspondence Address: WERNER DESMOND VAN JAARSUELDT, 21 CARLOTTA CRESCENT, WINNIPEG, MB, R3R2P4, CA

(21) Appl. No.: 10/612,682

(22) Filed: Jul. 3, 2003

(51) Int. Cl. A61M 5/00

(52) U.S. Cl. 604/117; 604/275

(57) ABSTRACT

The ear syringe tip as embodied in this filing would have soft, catheter like tip as compared to the hard inflexible tips of the older versions. The ear syringe tip would also have very smooth rounded edges as compared to the sharp edges of predecessors tips. Instead of being fixed onto a syringe this tip is removable and disposable in order to facilitate use and prevent disease transmission. The catheter can also be fitted with a clip on depth shield that serves as a guide as to the tip depth, prevents the tip being inadvertently inserted to deeply and prevents splash back of fluid onto the operator. The above features would greatly reduce the risk of injury and transmission of disease whilst increasing efficiency.

### CROSS-REFERENCE TO RELATED APPLICATIONS

U.S. Patent Documents

| D191088 | August 1961 | Anderson. | |
|---|---|---|---|
| D317203 | May 1991 | Walsh | D24/115. |
| 698511 | April 1902 | Jones. | |
| 1755754 | April 1930 | Wappler et al. | |
| 2879768 | March 1959 | Anderson. | |
| 3590722 | July 1971 | Leptrone | D24/115. |
| 3651808 | March 1972 | White | 604/39. |
| 3990448 | November 1976 | Mather et al. | |
| 4206756 | June 1980 | Grossan | 604/39. |
| 4258714 | March 1981 | Leopoldi et al. | 604/118. |
| 4300545 | November 1981 | Goodnow et al. | |

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF INVENTION

1. Field of Invention

The instant invention relates generally to the removal of debris and foreign objects from narrow tubular passages and specifically it relates to the removal of wax from the outer ear canal through the use of water lavage.

2. Description of Prior Art

The human ear canal constantly secretes wax that in some individuals unfortunately accumulates and finally totally obstructs the outer ear canal. The total obstruction leads to decrease hearing, the inability to inspect the tympanic membrane, tinnitus, and general discomfort. Many devices from common cotton swabs that only serve to impact wax further, to curets which can damage the outer ear canal and the tympanic membrane to ear syringes with rigid tips has been designed over the years to assist in the removal of wax from the outer ear canal. These devices all commonly share a hard or fairly rigid tip that is inserted into the outer ear canal that with improper use or due to sudden movement can cause significant trauma to the ear canal and tympanic membrane.

### SUMMARY OF THE INVENTION

The removal of wax from the outer ear canal should be a safe and simple procedure during which wax is easily removed without the danger of injury. Therefore what are presented in this instant invention are a safer and more effective as well as inexpensive device for removal of wax from the outer ear canal.

The ear syringe tip proposed is composed of a hard plastic kuegel shaped base, able to screw onto any standard disposable syringe at the wide end of the base through the use of a slightly beveled rim. At the narrow end of the kuegel shaped base is a curved, soft highly flexible catheter with frequent depth markings and and rounded tip. The catheter is soft and highly flexible to prevent injury to the outer ear canal during use and due to unexpected movement by both the user and and the ear canal. The tip is ideally slightly rounded to prevent any sharp edges scraping the outer ear canal. The catheter has depth markings to indicate the length inserted into the outer ear canal and is ideally of such a length as to be impossible to touch the tympanic membrane even it fully inserted. The last could be achieved be either leaving the catheter fairly short or by the attachment of a transverse brace at the appropriate depth. The catheter is curved so that the tip is at a ninety degree angle to the base in order that sudden movement by either the operator or ear canal would promote the catheter to automatically be withdrawn rather than unexpectedly be plunged deeper into the ear canal.

The ear syringe tip may be used with manual devices such as syringes or used as an attachment to automatic ear lavage pumps.

The ear syringe tip is to be safe due its curved design, soft catheter, round tip, depth markings and adjustable cross brace.

The ear syringe tip is to be hygienic and disposable due to low cost.

The ear syringe tip is to be easily adapted for use in adults, children, ears and other body cavities that might require such an object (such as the collection of vaginal fluid samples for forensic purposes).

The accomplishment of the above and related, is achieved by the invention as embodied by the attached drawings, although important to note that the drawings are illustrative only and serves only to help visual the general device. Therefore numerous changes are possible to the construction illustrated and described whilst still being within the scope of the claim.

### BRIEF DESRIPTION OF THE SEVERAL VIEWS OF THE DRAWINGS

FIG. 1 is a Side View of the Ear Syringe Tip, demonstrating the rounded tip, flexible catheter with depth shield and base with wings for attachment to a syringe

FIG. 2 is a Side View of The Ear Syringe Tip attached to a syringe, demonstrating the base attached to a syringe and the key claims of the ear syringe tip.

FIG. 3 is a Magnified Transverse View of the Rounded Catheter Tip

FIG. 4 is a Magnified Transverse View of the Depth Shield

FIG. 5 is a Magnified Transverse View of the screw on Base of the catheter, with wings to allow it to screw onto a syringe

FIG. 6 is a Magnified Front View of the Depth Shield, showing the grove on one side allowing it to be removed from the catheter on to be moved along the shaft to a desired depth

FIG. 7 is a Magnified Front View of the Base of the catheter, demonstrating a top view of the wings that allows the base to attach by a screwing motion onto a syringe

FIG. 8 is a Magnified Transverse View of a syringe with an abnormally long tip inserted into the outer ear canal, with the depth shield preventing the catheter from being inserted to deep as well as splash back. The syringe is partially filled with water and water and debris is seen dripping from the ear pinna.

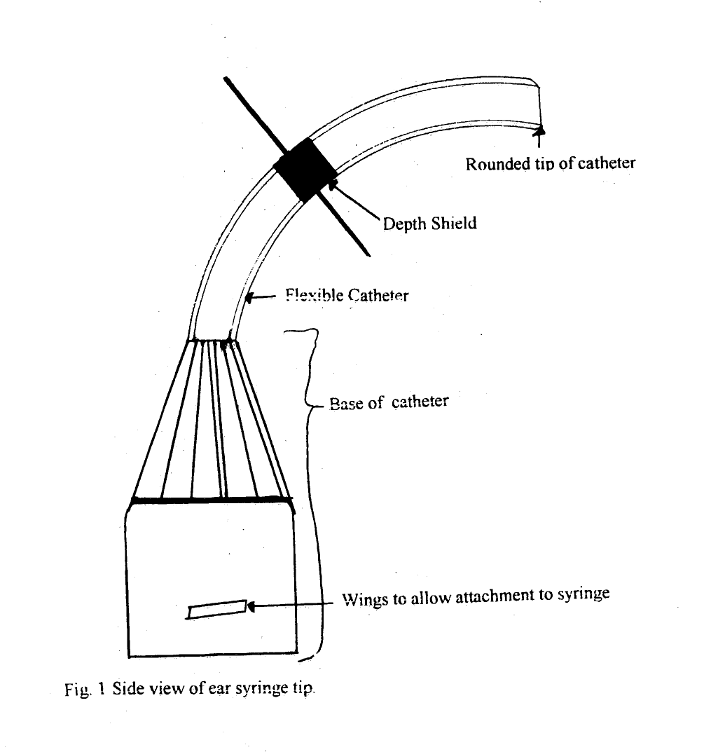

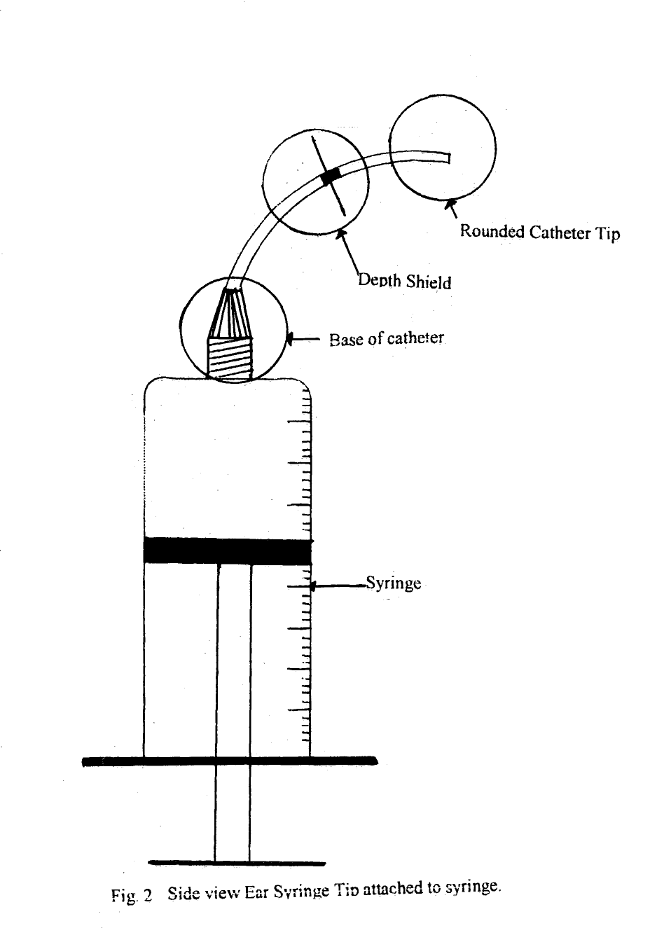

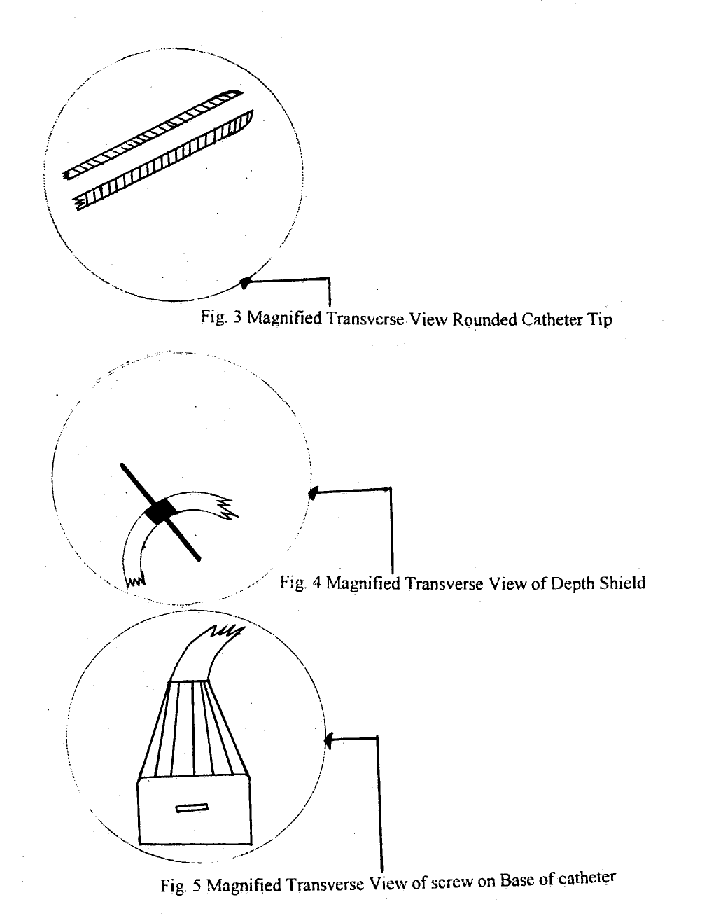

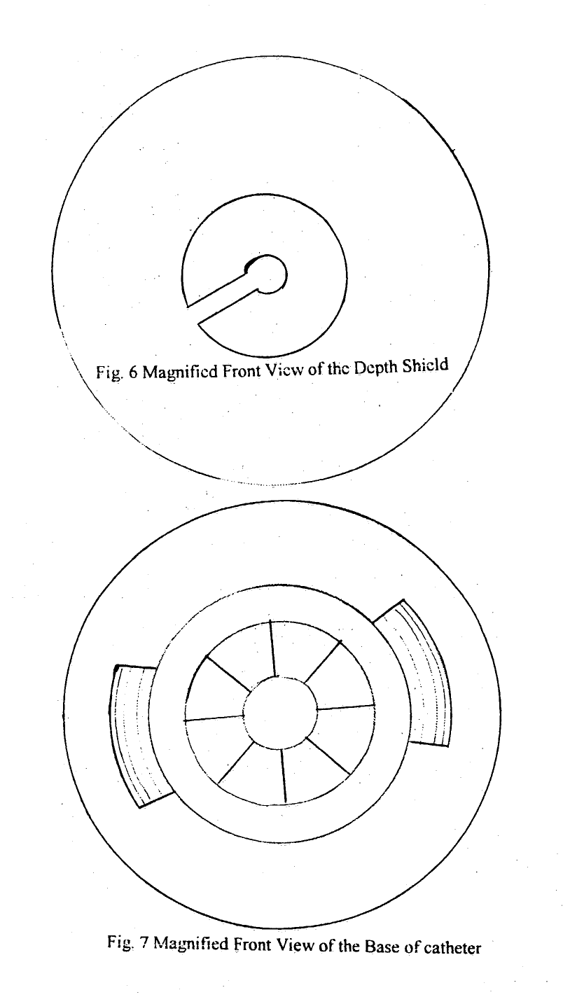

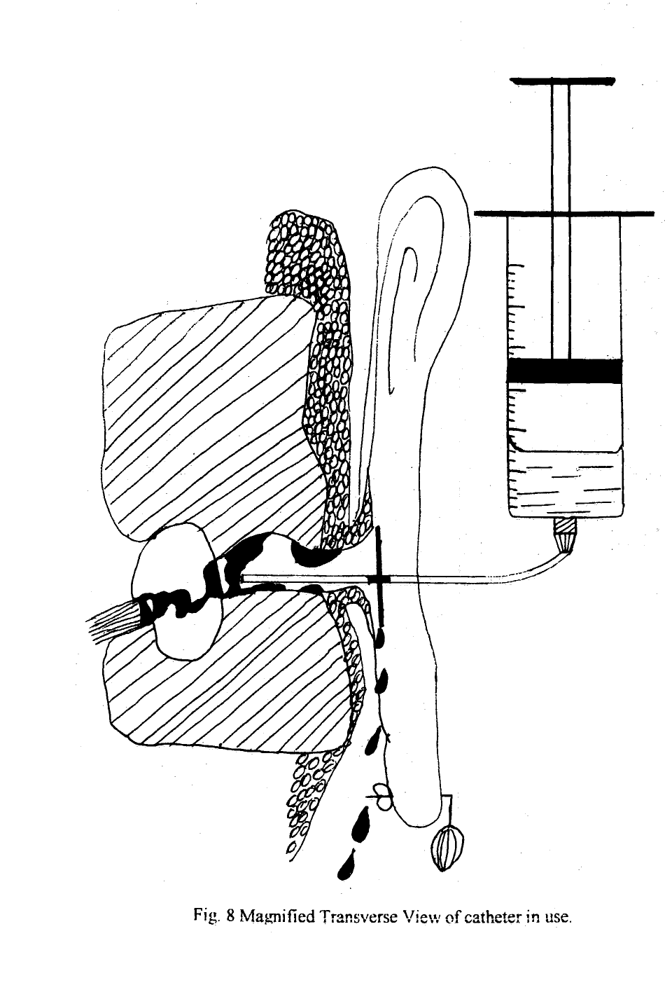

### DETAILED DESRIPTION OF INVENTION

The ear syringe tip is manufactured using simple plastics injection moulding similar as what is used for the manufacture of syringe needles. The tip consists however of a flexable selastic catheter rather than a stainless steel needle shaft though. The curvature is achieved by stretching the catheter over a sharp edge and the depth shield is a clip on plastic disc. The depth shield disc could be clipped on at any distance from the tip, as determined by the operator as safe.

This invention differs dramatically from prior inventions in as so far the catheter is made of a soft totally flexible plastic. This soft catheter will significantly mimise the risk of damage the ear drum in the event of the tip inadvertently pushed deeper into the ear canal than intended.

The further most tip as rounded shoulders to prevent any sharp edges that might scratch or irritate the ear canal. The catheter can be fitted with a depth shield that serve both as an indicator of what length of catheter is placed into the ear canal as well as preventing more of the catheter tubing to enter the canal in case of an accident and lastly to prevent splash back of water out of the ear canal.

The base of the catheter has to small wings allowing it to be easily screwed onto any disposable syringe. This feature enables the operator to remove the tip whilst filling the syringe and reattaching it afterwards as well as allowing tips to be separatedly manufactured, shipped, sold and fitted as is needed by the operator onto already available syringes instead of buying specific syringes.

The low cost of manufacture would enable the tips to be totally disposable which is preferable to the current re-use of ear syringes which could easily lead to the transfer of disease from person to person due to minor scratches caused by current syringes in the outer ear canal.

The use of the tip would involve the operator to fill a 60 ml disposable syringe with body temperature water and then with a twisting motion screwing the base of the catheter onto the tip of the syringe. The clip on depth shield is then clipped onto the catheter at a safe distance from the tip, so as to prevent the tip from being able to touch the tympanic membrane. The tip of the catheter is then inserted into the outer ear canal whilst holding the syringe flat and parallel to the patients head (preferably pointing downward/in the vertical position).

Whilst gently pulling the pinnae up and backwards to straighten the ear canal as much as possible the other hand is used in a manner normal to injecting any substance (the index and middle fingers braced against the chamber wings whilst the thumb depresses the plunger) to flush the water into the ear canal.

The force of the water can be controlled by the operator's amount of pressure on the syringe plunger and motion of the tip can be induced by gently wiggling the syringe. This should enable the water to loosen any wax or foreign bodies in the ear canal. The process is repeated until the desired effect is achieved.

1. An ear syringe tip comprising of:
a solid kuegel shaped base with a beveled wings able to screw onto a regular plastic disposable syringe;
with a curved, soft flexable catheter tip
with depth markers at regular intervals
with a depth shield attachment
and a rounded tip.
